(19) United States
(12) Patent Application Publication
(10) Pub. No.: US 2003/0113226 A1
(43) Pub. Date: Jun. 19, 2003

(54) Device and method for sterilizing or disinfecting waste

(76) Inventor: Robert Marcel Fliers, Voorburg (NL)

Correspondence Address: William B Patterson, Moser Patterson & Sheridan, Suite 1500, 3040 Post Oak Blvd, Houston, TX, 77056, US

(21) Appl. No.: 10/149,627

(22) PCT Filed: Dec. 14, 2000
(86) PCT No.: PCT/NL00/00916

(30) Foreign Application Priority Data
Dec. 14, 1999 (NL) 1013839

(51) Int. Cl. A01N 1/00; A61L 2/00

(52) U.S. Cl. 422/38; 422/1; 422/307

(57) ABSTRACT

A device for sterilizing or disinfecting waste that is contaminated with microorganisms, comprising a container which is capable of retaining the waste, means disposed in said container which are capable of pulverizing and/or crushing said waste, and driving means which are capable of driving said pulverizing and/or crushing means, wherein the driving means of the device comprise a combustion engine, which is capable of transmitting heat to the waste.

The invention relates to a device for sterilizing or disinfecting waste that is contaminated with microorganisms, comprising a container which is capable of retaining the waste, means disposed in said container which are capable of pulverizing and/or crushing said waste, and driving means which are capable of driving said pulverizing and/or crushing means.

A device of this kind is known from international patent application no. WO 95/03072. In this prior art device a container is filled with waste, for example hospital waste, after which a rotor fitted with blades is driven by an electric motor to a speed of about 1500 rpm. The blades pulverize and crush the hospital waste, whereby so much heat is generated by friction that the temperature of the waste rises to a suitable sterilizing or disinfecting temperature of 160-180° C., for example, after which the waste is maintained at this temperature for 20-30 minutes, whereby the rotor continues to rotate whilst at the same time water is sprayed onto the waste. Following this, the waste is cooled down and the manageable, pulverized waste can be discharged.

It has become apparent that this prior art way of heating the waste by means of friction energy is not an efficient way of reaching the desired temperature. The process takes relatively much time and requires a great deal of electricity. The crushing effect of the rotor is reached after a few minutes already, after that the rotor only needs to be rotated in order to generate sufficient heat.

Accordingly it is an object of the invention to provide a device for sterilizing or disinfecting waste that is contaminated with microorganisms, by means of which the waste is heated and pulverized and/or crushed in an efficient, quick and inexpensive manner.

In order to accomplish that objective, the driving means of the device comprise a combustion engine, which is capable of transmitting heat to the waste. Preferably, this is achieved in that the combustion heat is transmitted to the waste by means of exhaust gases. The term combustion engine is understood to mean an engine which is driven by a fuel. By supplying the combustion heat from a combustion engine to the waste the heating function and the pulverizing function of the device are more or less separated, and the heat for achieving the sterilizing and/or disinfecting temperature does not need to originate from the friction heat of the rotor blades alone but also from the heat produced by the engine, as a result of which a more effective, cheaper and quicker heating effect is obtained.

In a first preferred embodiment an exhaust of the combustion engine is connected to the container, in such a manner that the heat from the exhaust gases can be transmitted to the waste in that the exhaust gases from the combustion engine are introduced into the container. In this manner the set objectives are accomplished in a simple and efficient manner.

In a second preferred embodiment an exhaust of the combustion engine is connected to a heat exchanger, in which the exhaust gases can transmit the combustion heat to a pipe containing a medium, which pipe is connected to the container, in such a manner that the heat from the medium can be transmitted to the waste in that the medium is introduced into the container. The use of a heat exchanger leads to a more uniform and more controlled supply of heat to the container.

Preferably, the pulverizing and/or crushing means comprise a rotor, which is fitted with blades that are capable of pulverizing and/or crushing the waste in an efficient manner.

The invention furthermore relates to a method for sterilizing or disinfecting waste that is contaminated with microorganisms, wherein the waste is placed into a container, pulverizing and/or crushing means pulverize and/or crush the waste, which pulverizing and/or crushing means are driven by driving means, wherein the waste is heated to a temperature suitable for sterilization and/or disinfection, wherein said driving means comprise a combustion engine, and wherein combustion heat from the combustion engine is transmitted to the waste.

The invention will now be explained in more detail by means of exemplary embodiments, with reference being made to the drawings, wherein:

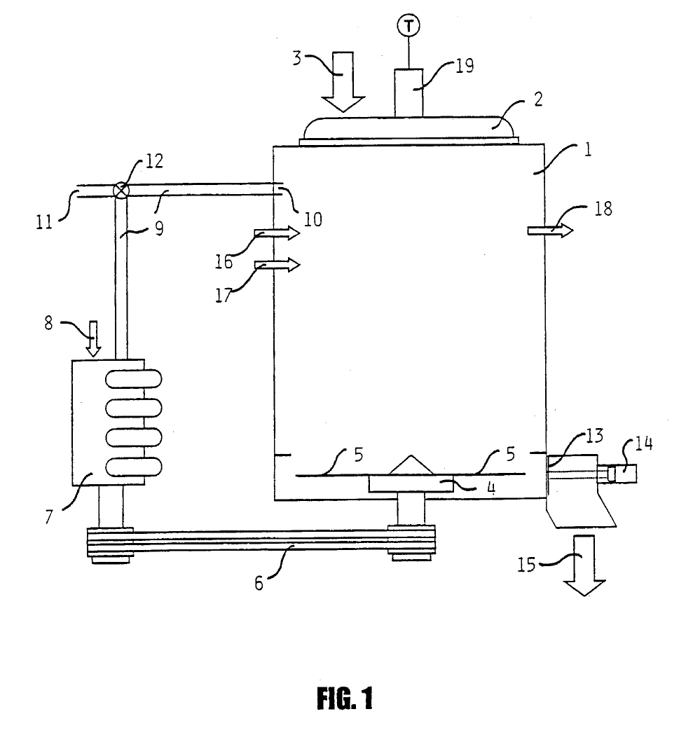

FIG. 1 schematically shows a first exemplary embodiment of a device for sterilizing or disinfecting waste; and

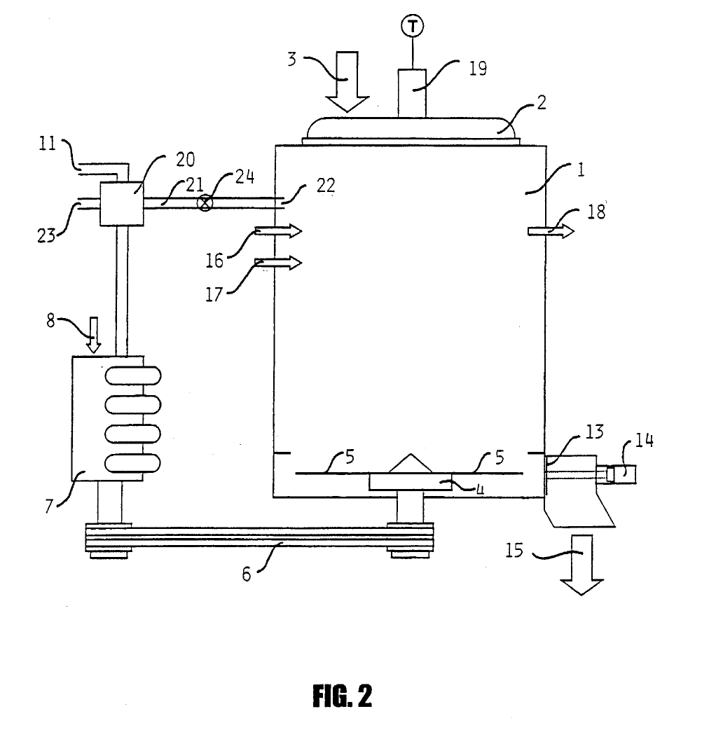

FIG. 2 schematically shows a second exemplary embodiment of a device for sterilizing or disinfecting waste.

According to FIG. 1 a device for sterilizing or disinfecting waste contaminated with microorganisms, for example from a hospital, comprises a container 1 which is closed by a cover 2, through which the waste can be deposited into container 1 (symbolically indicated by arrow 3). The container has a diameter of 0.35-2 m. Present inside the container is a rotor 4 with a vertical shaft, which rotor is fitted with 2 blades or knives 5. Rotor 4 can be rotated at a high rotational speed, for example a speed of 1500 rpm, whereby the blades or knives 5 make a circular movement at a distance of about 50-150 mm above the bottom of container 1.

The rotor 4, which is bearing-mounted in the bottom of container 1, is driven by a combustion engine 7 via a transmission 6, which may for example be a belt transmission, gear transmission or any other known transmission. Combustion engine 7 may also be directly connected to the rotor 4. Combustion engine 7 is fed by a fuel, for example petrol or diesel oil (symbolically indicated by arrow 8) and it has variable speed. A first end 10 of the exhaust 9 of the combustion engine opens into the upper part of the container 1. A second end 11 of exhaust 9 opens into the atmosphere surrounding the device. The exhaust gases can be led to the first or to the second end by means of a valve 12.

Present in the circumference of the container 1, near the bottom side thereof, is a valve 13, which can be opened and closed by means of a hydraulic cylinder 14. In the open position the processed waste can flow out of container 1 (symbolically indicated by arrow 15) and be collected in a waste container, for example.

Furthermore connected to container 1 is a supply line, through which water can be sprayed into container 1 (symbolically indicated by arrow 16). In addition to that another supply line is connected to container 1, through which a disinfectant, for example hypochlorite, can be sprayed into container 1 (symbolically indicated by arrow 17). Likewise connected to vessel 1 is a discharge line, through which the humid vapours from the sterilization or disinfection process can be discharged (symbolically indicated by arrow 18). Said vapours are cleaned and heated before being emitted into the environment. The exhaustion through the discharge line results in a continuous underpressure being generated in the container, which ensures that hazardous microorganisms and other substances cannot exit from container 1 due to leakage. In addition, said underpressure ensures that the exhaust gases from the combustion engine 7 are sucked into container 1.

Mounted in cover 2 is a temperature sensor 19, which measures the temperature inside the container 1. Temperature sensor 19, along with other components, is connected to a control computer (not shown), which is capable of operating inter alia fuel supply line 8, valve 12, hydraulic cylinder 14, water supply line 16, disinfectant supply line 17 and vapour discharge line 18.

The contaminated waste is placed into container 1 in the open position of cover 2, as is indicated by arrow 3, for the purpose of sterilizing and disinfecting said waste, after which the cover 2 is closed. Then the control computer starts the combustion engine 7, as a result of which the rotor 4 is rotated at a speed of 1500 rpm. The knives or blades 5 pulverize and/or crush the waste, whereby heat is generated as a result of the friction between the knives or blades 5 and the waste. At the same time the exhaust gases from the combustion engine 7 are introduced into container 1, as a result of which additional heat is transmitted to the waste. First the temperature is quickly raised to about 105° C. in this manner, at which temperature the waste is maintained for about 10 minutes in order to kill all bacteria that are present. A hypochlorite solution may be sprayed onto the waste through supply line 17 so as to boost this process. During this stage and other stages of the process, whereby no additional supply of heat is desired, the valve 12 is set so that the exhaust gases are carried to end 11. Then the temperature in container 1 is raised further to about 155° C., at which temperature the waste is maintained for about 5 minutes in order to kill all viruses that are present. During this stage water is sprayed on to the waste through supply line 16, as a result of which steam is produced, which steam ensures that an effective sterilization takes place. Following this, the cleaned, pulverized and crushed waste is cooled down by spraying more water onto said waste, after which valve 13 is opened and the waste is removed from container 1 in the direction indicated by arrow 15 by the centrifugal force exerted by the rotor.

In another embodiment according to FIG. 2 the exhaust gases are passed through a heat exchanger 20 and they are emitted through the end 11 of exhaust 9. A pipe 21 has a first opening 22 in container 1 and a second opening 23 in the atmosphere surrounding the device. As a result of the underpressure in container 1 air from the surrounding atmosphere flows into container 1, whereby said air takes over heat from the exhaust gases in heat exchanger 20. If additional supply of heat in container 1 is undesirable, valve 24 can be closed. For the rest the device operates in a similar manner as the device according to FIG. 1.

1. A device for sterilizing or disinfecting waste that is contaminated with microorganisms, comprising a container which is capable of retaining the waste, means disposed in said container which are capable of pulverizing and/or crushing said waste, and driving means which are capable of driving said pulverizing and/or crushing means, characterized in that the driving means of the device comprise a combustion engine, which is capable of transmitting heat to the waste.

2. A device according to claim 1, wherein the combustion heat is transmitted to the waste by means of exhaust gases.

3. A device according to claim 2, wherein an exhaust of the combustion engine is connected to the container, in such a manner that the heat from the exhaust gases can be transmitted to the waste.

4. A device according to claim 2, wherein an exhaust of the combustion engine is connected to a heat exchanger, in which the exhaust gases can transmit the combustion heat to a pipe containing a medium, which pipe is connected to the container, in such a manner that the heat from the medium can be transmitted to the waste.

5. A device according to any one of the preceding claims, wherein the pulverizing and/or crushing means comprise a rotor, which is fitted with blades that are capable of pulverizing and/or crushing the waste.

6. A method for sterilizing or disinfecting waste that is contaminated with microorganisms, wherein the waste is placed into a container, pulverizing and/or crushing means pulverize and/or crush the waste, which pulverizing and/or crushing means are driven by driving means, wherein the waste is heated to a temperature suitable for sterilization and/or disinfection, wherein said driving means comprise a combustion engine, and wherein combustion heat from the combustion engine is transmitted to the waste.

7. A method according to claim 6, wherein at least part of the combustion heat is transmitted to the waste in that exhaust gases from the combustion engine are introduced into the container.

8. A method according to claim 6, wherein at least part of the combustion heat is transmitted to the waste in that exhaust gases from the combustion engine transmit the combustion heat to a medium in a heat exchanger, which medium is introduced into the container.

9. A method according to claim 6, 7 or 8, wherein said waste is heated to the temperature suitable for sterilization and/or disinfection both by the combustion heat from the combustion engine and by the friction heat obtained from pulverizing and/or crushing the waste.
